(19) United States
(12) Patent Application Publication
(10) Pub. No.: US 2010/0018469 A1
(43) Pub. Date: Jan. 28, 2010

(54) Identification and Sampling Device for Animals

(75) Inventor: Richard Gottschling, Mittenwald (DE)

Correspondence Address: ALLEMAN HALL MCCOY RUSSELL & TUTTLE LLP, 806 SW BROADWAY, SUITE 600, PORTLAND, OR, 97205-3335, US

(73) Assignee: GEPE-GEIMUPLAST GMBH, Farchant (DE)

(21) Appl. No.: 12/505,288

(22) Filed: Jul. 17, 2009

(30) Foreign Application Priority Data
Jul. 25, 2008 (DE) 10 2008 034 677.2

(51) Int. Cl. A01K 29/00; A61B 10/00

(52) U.S. Cl. 119/174; 600/567

(57) ABSTRACT

An identification and sampling device, in particular for animals, comprises an ear tag and a sample container. The ear tag comprises a male part, which includes a shaft and a tip, and a female part, which can be latched with the male part. The sample container includes a receiving space for a sample and a punch.

### CROSS REFERENCE TO RELATED APPLICATION

This application claims priority to German Patent Application No. 10 2008 034 677.2, filed Jul. 25, 2008, which is hereby incorporated by reference in its entirety for all purposes.

### TECHNICAL FIELD

This invention relates to an identification and sampling device for marking an animal and for taking and storing a tissue sample of an animal. This invention furthermore relates to a pair of tongs for connecting an ear tag and for taking a tissue sample.

### BACKGROUND AND SUMMARY

Animals, in particular cattle, must be marked for various purposes. For marking an animal, ear tags can be used, which include a male part and a female part. Such ear tag is known from DE 20 2004 011 020 U1. The male part includes a shaft and a tip. The female part can latched with the male part. When applying the ear tag, the male part and the female part are positioned on different sides of the ear of the animal. When connecting the parts, the tip of the male part pierces the tissue. When the tip has completely penetrated the female part, it latches with the same. The ear or other tissue part of the animal then is located in the vicinity of the shaft of the male part.

In many applications, it is required to take a tissue sample of an animal, in particular of a cow. Devices for taking a tissue sample of an animal are known from the above-mentioned DE 20 2004 011 020 U1, from EP 1 014 861 B1 and from EP 1 088 212 B1.

It is the object of the invention to propose an improved identification and sampling device as indicated above. Another object of the invention consists in proposing improved tongs for connecting an ear tag and for taking a tissue sample.

In accordance with the invention, this object is solved in an identification and sampling device comprising an ear tag and a sample container. The ear tag comprises a male part and a female part. The male part includes a shaft and a tip. The female part can be latched with the male part. The sample container includes a receiving space for the sample and a punch. When applying the ear tag and when taking the tissue sample, the ear tag and the sample container are separated from each other.

Advantageous developments are further described herein.

In one example, the sample container includes a supporting member. The supporting member serves to support the sample container on a pair of tongs. In accordance with the invention, a pair of tongs is understood to be an applicator for the ear tag. By means of the supporting member, the sample container can be supported on the tongs.

In one example, the supporting member can be separated from the sample container. The supporting member can serve to support the sample container in the tongs before taking the sample. During or after taking the sample, the supporting member can be separated from the sample container. Separating the supporting member from the sample container is effected by closing the tongs.

Advantageously, an end cap for the sample container is provided on the supporting member. Upon taking the sample, the sample container can be closed by the end cap.

In accordance with another advantageous development, the sample container includes a stop. The stop is located on the outside of the sample container. Advantageously, the stop serves to limit the movement of the sample container in the tongs.

The male part and/or the sample container and/or the female part can be insertable in a pair of tongs. The male part on the one hand and the sample container and/or the female part on the other hand are insertable in different arms of a two-arm pair of tongs.

Advantageously, the female part can be put against, or abut, the sample container. In use of the identification and sampling device, the male part and the sample container can be inserted in the tongs. The female part can be put against the sample container, so that then all three parts are correctly positioned relative to each other and to the tongs.

The object underlying the invention is solved in a pair of tongs for connecting an ear tag of the invention and for taking a tissue sample by means of a sample container of the invention in that the tongs include a receptacle for the male part and a receptacle for the sample container.

Advantageously, the tongs include a receptacle for the female part. Then, it is possible to also use the tongs when merely an ear tag should be attached, but no sampling should be affected.

### BRIEF DESCRIPTION OF FIGURES

An embodiment of the invention will be explained in detail below with reference to the attached drawing, in which:

FIG. 1 shows a partly sectional side view of a pair of tongs in which a sample container is inserted,

FIG. 2 shows a partly sectional side view of the tongs of FIG. 1, in which a sample container and a female part of an ear tag are inserted,

FIG. 3 shows a partly sectional side view of the tongs of FIGS. 1 and 2, in which a male part of an ear tag is inserted in addition,

FIG. 4 shows the sample container, the female part and the male part of FIG. 3 in an enlarged representation,

FIG. 5 shows the parts of FIG. 4 after piercing the ear and taking a sample,

FIG. 6 shows the parts of FIGS. 4 and 5 after connecting the ear tag,

FIG. 7 shows the sample container in a side view and in a top view,

FIG. 8 shows a section through the sample container of FIG. 7 before closing the same with an end cap,

FIG. 9 shows the sample container of FIGS. 7 and 8 after closing the same with the end cap,

FIG. 10 shows part of the tongs with inserted male part and female part without sample container in a sectional view corresponding to FIG. 4, and

FIG. 11 shows the male part and the female part of FIG. 10 after connecting.

### DETAILED DESCRIPTION

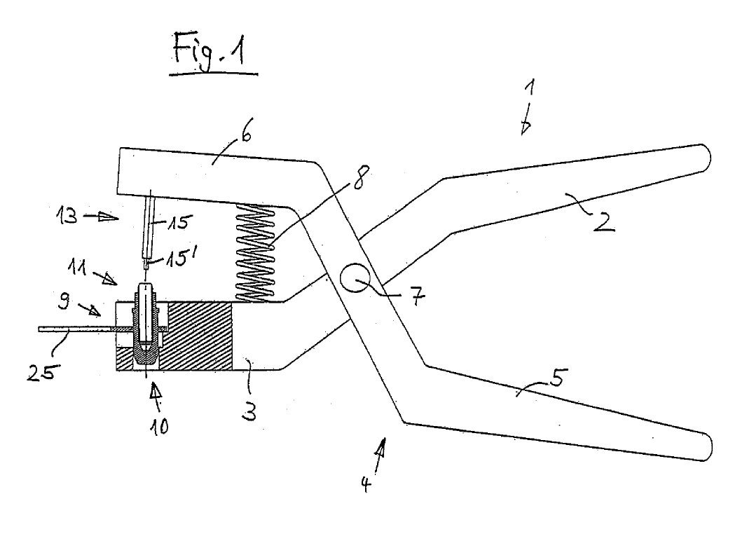

FIG. 1-3 show a pair of tongs, i.e. an applicator for an ear tag, in a side view. The tongs comprise a first lever 1 with an upper handle part 2 and a lower arm 3 and a second lever 4 with a lower handle part 5 and an upper arm 6. The levers 1 and 4 are pivotally connected with each other by an axle 7 and biased towards the open position by a compression spring 8, which is provided between the arms 3, 6.

The lower arm 3 of the tongs includes a receptacle 9 for a sample container 10 and a receptacle 11 for a female part 12. The female part 12 is put against the sample container 10, namely such that the hole of the female part 12 is put onto the upper end of the sample container 10. The upper arm 6 of the tongs includes a receptacle 13 for a male part 14. The receptacle 13 is configured as a pin 15. The male part 14 comprises a shaft 16, a tip 17 and a flat part 18, on which a mark can be provided, for instance an alphanumeric text and/or a bar code and/or one or more other marks and/or data carriers. The female part 12 likewise comprises a flat part 19, on which marks and/or data carriers can likewise be provided.

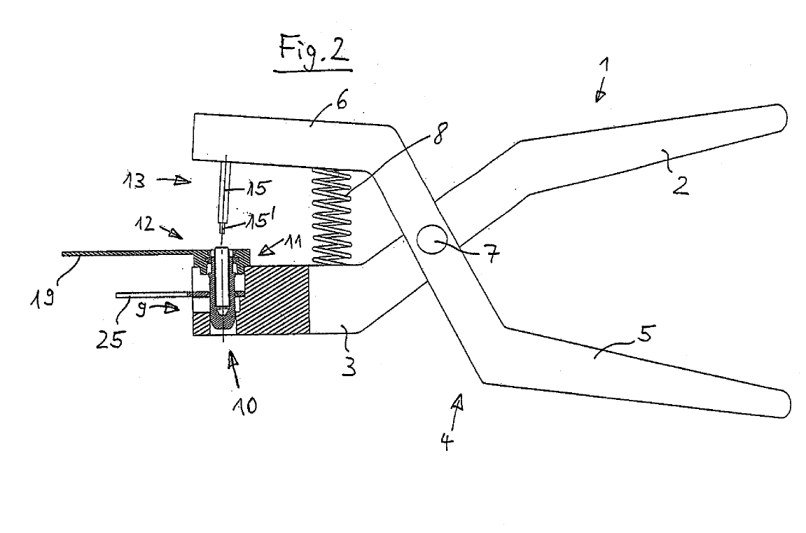

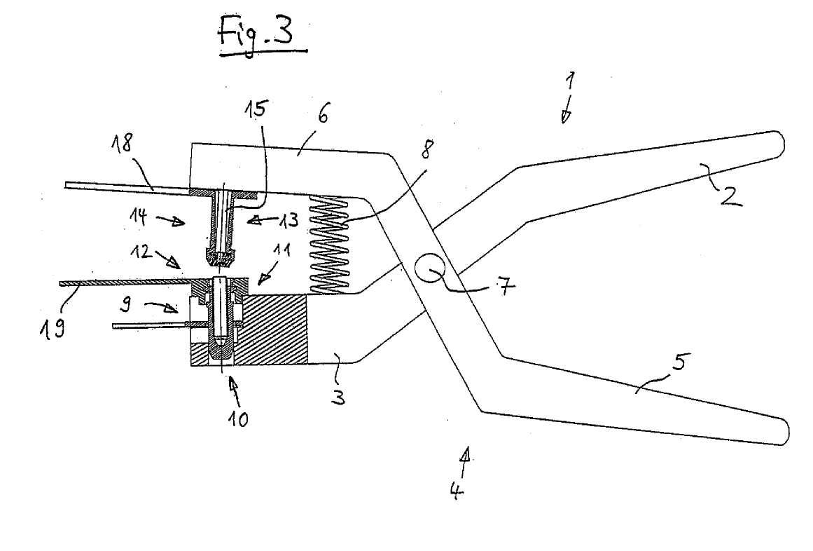

FIG. 1 to 3 show the time sequence when using the identification and sampling device. First of all, the sample container 10 is inserted into the receptacle 9 in the lower arm 3 of the tongs, as shown in FIG. 1. Subsequently, the female part 12 is inserted into the receptacle 11 in the lower arm 3 of the tongs, as shown in FIG. 2. At the same time, the female part 12 is put against the sample container 10. This is accomplished in that the female part 12 is put onto the sample container 10 from above. In the meantime, or before or after, the male part 14 is inserted into the receptacle 13 in the upper arm 6 of the tongs, as shown in FIG. 3. In the representation of FIG. 3, sample container 10, female part 12 and male part 14 are correctly positioned relative to each other and to the tongs.

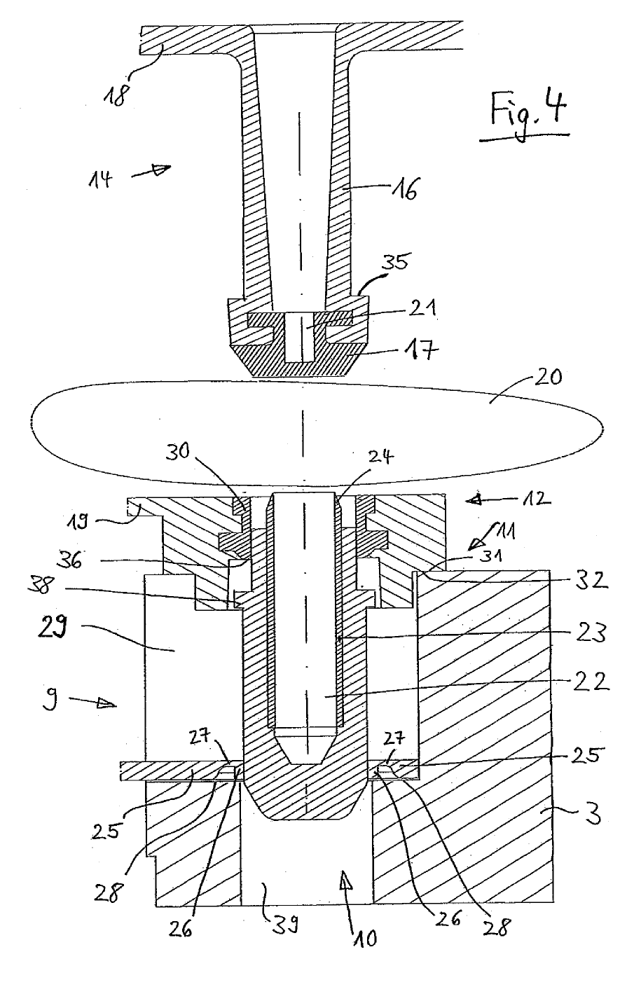

FIG. 4 shows the sample container 10, the female part 12 and the male part 14 in the position of FIG. 3 in an enlarged representation. Part of the ear 20 of an animal, for instance of a cow, is located between the tip 17 of the male part 14 on the one hand and the female part 12 and the upper end of the sample container 10 on the other hand.

The flat part 18 and the shaft 16 of the male part 14 are made of plastics. The tip 17 is made of a rigid plastic material and/or of metal or a composite material. The shaft 16 is hollow, so that the pin 15 of the receptacle 13 can be accommodated therein. In the tip 17, a depression 21 is provided, which forms a seat for the end 15′ of the pin 15.

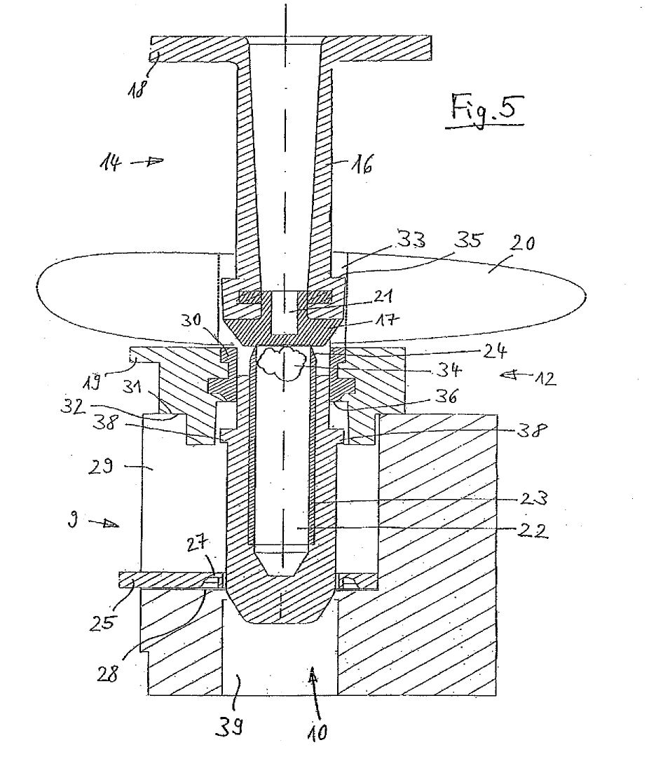

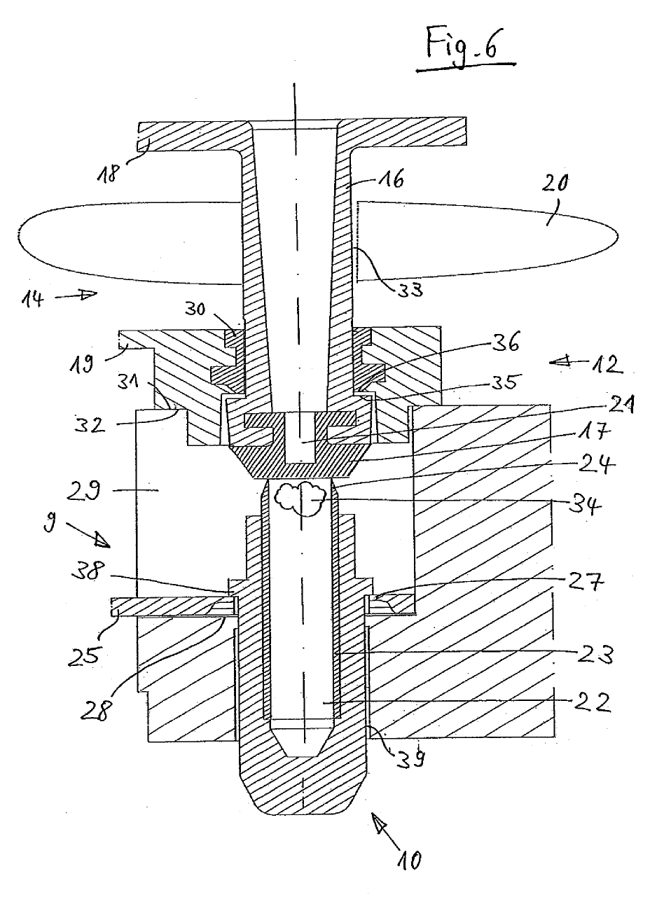

The sample container 10 comprises a receiving space 22 for a sample 34 (FIGS. 5 and 6) and a punch 23. The sample container 10 is closed at the bottom and open at the top. The punch 23 constitutes a sleeve, which defines part of the receiving space 22 and at its upper end has a cutting edge 24, which protrudes from the top of the sample container 10.

The sample container 10 furthermore includes a supporting member 25, which is connected with the sample container 10 in its lower region. The connection is effected via a sleeve member 26 connected with the sample container 10, which is adjoined by one or more thin webs 27 which then pass into a broader region. The receptacle 9 for the sample container 10 includes a shoulder 28, on which rests the supporting member 25. The sample container 10 can laterally be inserted into the receptacle 9 through a slot 29.

The female part 12 is made of plastics. It includes a counter-bearing 30 for the tip 17 of the male part 14. The counter-bearing 30 substantially is sleeve-shaped. It is made of a rigid plastic material and/or of metal or a composite material and connected with the female part 12. The counter-bearing 30 includes a substantially circular cylindrical through hole.

On its side facing the receptacle 11, the female part includes a shoulder 31, which rests on a shoulder 32 of the receptacle 9 in the lower arm 3 of the tongs. The inside diameter of the counter-bearing 30 of the female part 12 substantially corresponds to the outside diameter of the sample container 10 in its upper region.

When the tongs are actuated by pressing together the handle parts 2, 5, their arms 3, 6 also are moved towards each other. Due to this movement, the tissue of the ear 20 is pierced by the tip 17 of the male part 14, so that a passage 33 is formed in this tissue. In this way, the end of the tip 17 of the male part 14 facing the female part 12 and the sample container 10 gets in contact with the cutting edge 24 of the punch 23 of the sample container 10, as shown in FIG. 5. During said movement, the tip 17 has taken a sample 34 from the ear 18, which as a result of this movement is introduced into the receiving space 22 of the sample container 10.

Due to further closing of the tongs, the male part 14 is moved further downwards. The tip 17 penetrates the counter-bearing 30. On the back of the tip 17, a shoulder 35 pointing to the inside is provided in the shaft 16, which upon penetrating the counter-bearing 30 latches with a shoulder 36, which is formed by the lower end of the counter-bearing 30. In this way, the male part 14 latches with the female part 12, as shown in FIG. 6.

During this downward movement, the tip 17 urges the cutting edge 24 of the punch 23 and with the same the sample container 10 downwards into the receptacle 9. During this movement, the webs 27 of the supporting member 25 are torn off. Upon rupture of the webs 27, the sample container 10 can be moved further downwards into the receptacle 9, as is likewise shown in FIG. 6. To limit this movement, the sample container 10 includes a stop 38. The stop 38 is provided on the outside of the sample container 10. In the end position, the stop 38 rests on the supporting member 25.

FIG. 6 shows the position of the parts with the tongs closed. When the tongs are opened again, the ear tag, which consists of the female part 12 and the male part 14 latched with the same, can be detached from the tongs. Furthermore, it then is possible to remove the sample container 10, in which the sample 34 is disposed, from the receptacle 9 of the tongs.

The receptacle 9 for the sample container comprises a lower hole or through hole 39, whose diameter is at least slightly larger than the outside diameter of the sample container 10, in order to provide for the movement of the sample container 10 into this hole 39, as shown in FIG. 6. The receptacle 9 furthermore comprises the slot 29 and the shoulder 28. The receptacle 11 for the female part 12 comprises the shoulder 32.

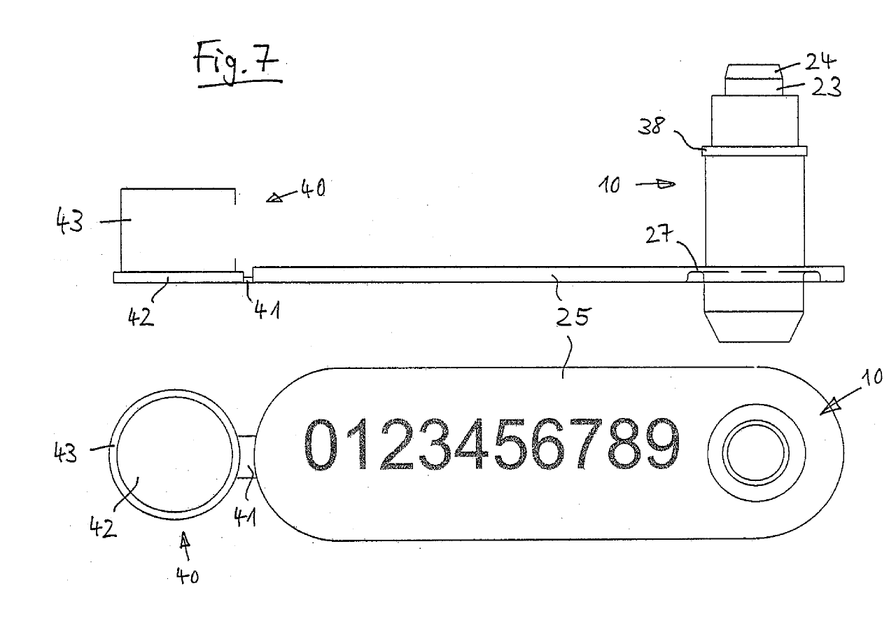

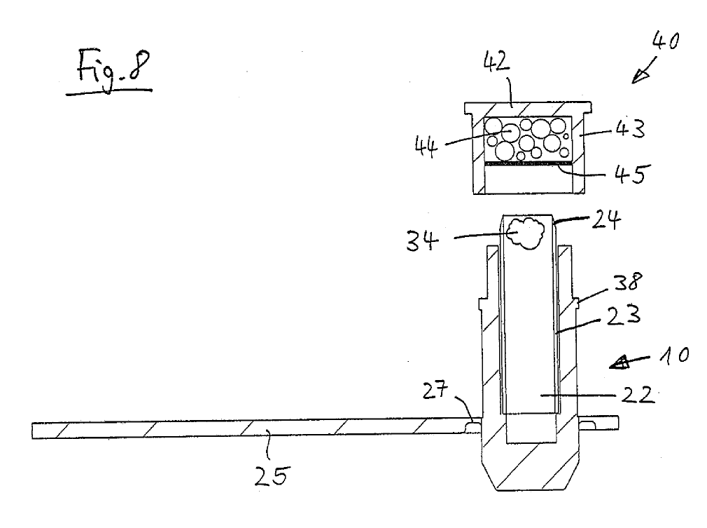

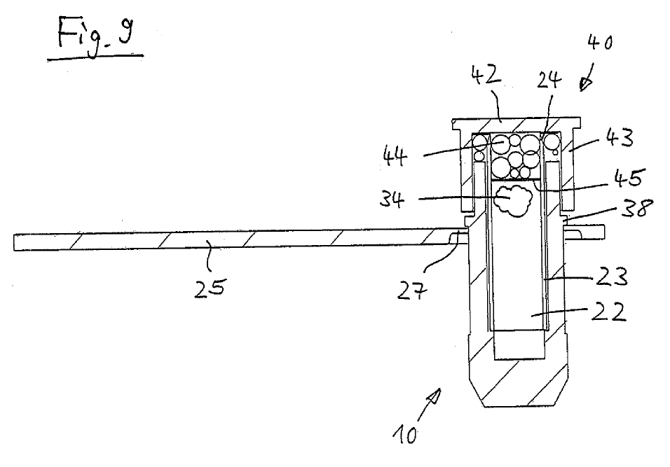

As can be taken from FIG. 7 to 9, an end cap 40 for the sample container 10 is provided on the supporting member 25. At its end facing away from the sample container 10, the end cap 40 is connected with the supporting member 25 via a tear-off web 41. As shown in FIGS. 8 and 9, the end cap 40 consists of a bottom 42 and a cylindrical wall 43. In the end cap 40, a chemical agent 44 is present, which is sealed between the bottom 42, the cylindrical wall 43 and a membrane 45. The membrane 45 is attached to the cylindrical wall 43. When the end cap is put onto the upper open end of the sample container 10, the cutting edge 24 of the punch 23 pierces the membrane 45, as shown in FIG. 9. The chemical agent 44 remains sealed between the bottom 42, the membrane 45 and a part of the side wall 43 of the punch 23. From there, it nevertheless can act on the sample 34. The chemical agent 44 can be present in granular, flowable form or in liquid, gel-like form or in some other form. In particular, it is an agent which has a preservative effect on the sample 34.

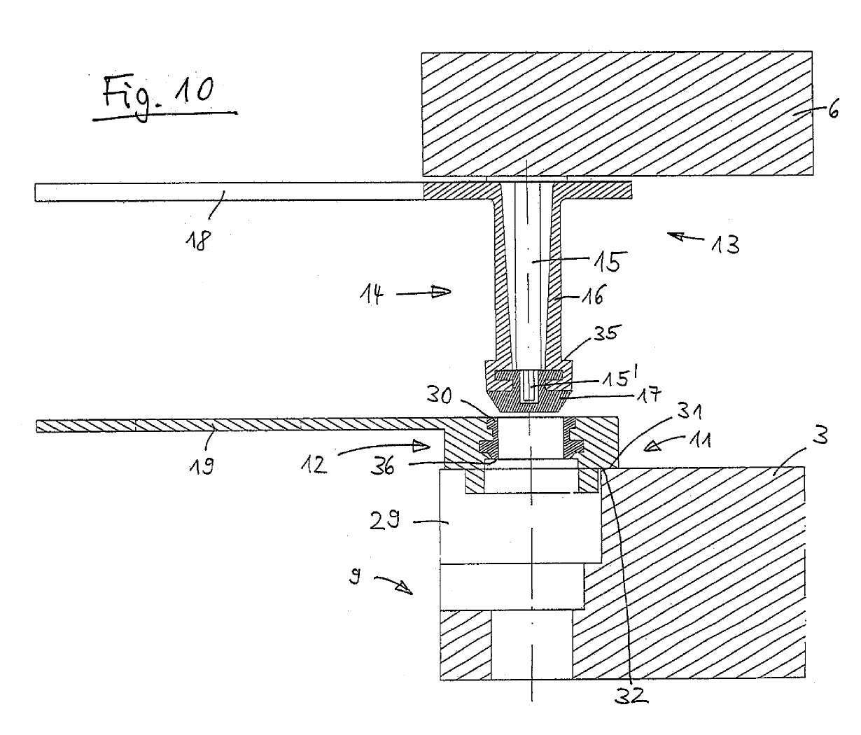

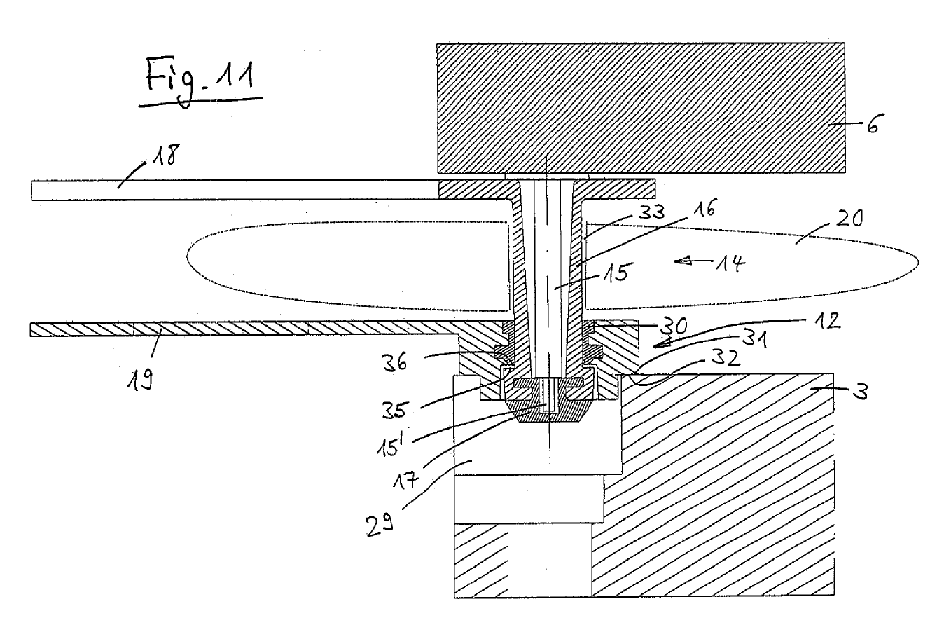

FIGS. 10 and 11 show that the tongs can also be used without sample container. When sampling is not required, the female part 12 is inserted into the receptacle 11, and the male part 14 is inserted into the receptacle 13, as shown in FIG. 10. Thereafter, the tongs can be closed, so that the tip 17 penetrates the counter-bearing 30 and latches with the same, as shown in FIG. 11. In this way, the ear tag is made, which consists of the female part 12 and the male part 14. By opening the tongs, the ear tag is released. It then remains in the ear 20 of the animal.

The female part 12, the male part 14 and the sample container 10 can include marks and/or data carriers. Advantageously, these parts include the same mark and/or a data carrier with the same data. It then is ensured that the sample 34 in the sample container 10 can unambiguously be allocated to a specific ear tag 12, 14.

By means of this invention, an identification and sampling device for animals is created, which comprises a two-part ear tag and a sample container constituting a separate part. The sample container can have the same identification as the ear tag. In one example, the ear tag consists of a female part and a male part. The sample container includes a punch, which can consist of a ground tube, in particular made of metal. For taking a sample, the sample container is inserted into the tongs. The male part is inserted into the other arm of the tongs. The female part is put onto the sample container, whereby it can be centered. The female part can loosely be put onto the sample container. It is, however, also possible to provide a clamping fit between the sample container and the female part. When closing the tongs, the tip of the male part presses the ear of the animal against the punch of the sample container. In the process, a piece of tissue is pushed into the internally hollow punch as a sample. Thereafter, the male part latches with the female part, whereby the ear tag is formed. At the same time, the sample container is again separated from the female part. It remains in the tongs, from which it can be removed again after opening the tongs.

In still another example, another aspect of the invention may include a method of simultaneously attaching an ear tag to an animal while collecting a tissue sample, the tissue sample collected via the eat tag attachment. The attaching may include inserting a male part, which includes a shaft and a tip, through the animal's ear, and engaging the male part with a female part to latch with the male part. The female part may include a sample container, the method further comprising receiving the tissue sample in the sample container, wherein upon attaching, the male part latches with the female part, forming the ear tag. At the same time, the sample container is separated from the female part and remains in the tongs, from which it can be removed again after opening the tongs.

1. An identification and sampling device, comprising
an ear tag with a male part, which includes a shaft and a tip, and with a female part, which is latchable with the male part,
and a sample container, which includes a receiving space for the sample and a punch.

2. The identification and sampling device according to claim 1, wherein the sample container includes a supporting member.

3. The identification and sampling device according to claim 2, wherein the supporting member is separable from the sample container.

4. The identification and sampling device according to claim 3, wherein an end cap for the sample container is provided on the supporting member.

5. The identification and sampling device according to claim 1, wherein the sample container includes a stop.

6. The identification and sampling device according to claim 1, wherein the male part is insertable into a pair of tongs.

7. The identification and sampling device according to claim 1, wherein the sample container is insertable into a pair of tongs.

8. The identification and sampling device according to claim 1, wherein the female part is insertable into a pair of tongs.

9. The identification and sampling device according to claim 1, wherein the female part is positioned to abut the sample container.

10. The identification and sampling device according to claim 1, wherein the sample container has the same identification code as the ear tag, and wherein the sample container is separable from the female part.

11. Tongs for connecting an ear tag and for taking a tissue sample via a sample container included in an identification and sampling device, the device further having a male part comprising a receptacle for the male part and a receptacle for the sample container.

12. The tongs according to claim 11, further including a receptacle for a female part.

13. An identification and sampling device for an animal, comprising:
a two-part ear tag having a first identification code, the two-part ear tag comprising a female part and a male part latchable with one another upon insertion into the ear, the insertion generating a tissue sample from the animal's ear; and
a sample container, where the container is a separate part from the two-part ear-tag, the sample container having a second identification code which matches the first code of the ear tag, the sample container including an internally hollow punch for capturing the tissue sample.

14. The device of claim 13, wherein the punch includes a metal ground tube.

15. The device of claim 14, wherein the punch includes a cutting edge which protrudes from a top of the sample container.
